(19) United States
(12) Patent Application Publication
(10) Pub. No.: US 2005/0232905 A1
(43) Pub. Date: Oct. 20, 2005

(54) Use of peripheral blood cells for cardiac regeneration

(76) Inventor: Edward T.H. Yeh, Houston, TX (US)

Correspondence Address: BAKER BOTTS L.L.P.;PATENT DEPARTMENT, 98 SAN JACINTO BLVD., SUITE 1500, AUSTIN, TX, 78701-4039, US

(21) Appl. No.: 11/091,149

(22) Filed: Mar. 28, 2005

Related U.S. Application Data
(60) Provisional application No. 60/557,101, filed on Mar. 26, 2004.

(51) Int. Cl. A61K 48/00; C12N 5/08

(52) U.S. Cl. 424/93.21; 435/372

(57) ABSTRACT

The present invention concerns methods of improving cardiac function in cardiac tissues of a recipient host by delivery of peripheral blood derived stem cells. The present invention also describes methods of stimulating cardiac tissue regeneration in a recipient host. Methods of treating a diseased cardiac tissue in a recipient host, as well as methods of obtaining a differentiated cardiac cell. Cardiac tissues subjected to these methods may be diseased or damaged, for example, by infarction, ischemia, chemotherapy, or infection. Peripheral blood derived stem cells used in the invention form new cardiomyocytes and also may form smooth muscle and endothelial cells.

### PRIORITY CLAIM

The present invention claims priority to U.S. Provisional Patent Application Ser. No. 60/557,101, filed Mar. 26, 2004 titled “Use of Peripheral Blood Cells for Cardiac Regeneration”, the entirety of which is also incorporated by reference herein.

### BACKGROUND

The present invention generally relates to methods of improving cardiac function in a recipient host with cardiac tissue damage. More specifically, the present invention relates to methods of stimulating cardiac tissue regeneration through the introduction of peripheral blood derived stem cells into a recipient host.

Cardiac dysfunction as a result of damaged myocardial tissue may occur after myocardial infarction (MI), chemotherapy-induced damages, or other injuries leading to heart failure. Once damaged, a process referred to as cardiac remodeling may occur. During this process, cardiomyocytes are gradually replaced by fibroid nonfunctional tissue. Regeneration of damaged myocardial tissue has been attempted using various stem cell populations, but has required significant manipulation.

A stem cell is generally considered to be a cell from which other types of cells may develop. Stem cells may give rise to one or more lineage-committed cells, some of which are also stem cells, that in turn give rise to various types of differentiated tissues. Such stem cells generally constitute a small percentage of the total number of cells present in the body and vary based on their relative level of commitment to a particular lineage. Stem cells have the ability to produce differentiated cell types and may therefore may be useful for replacing the function of aging or failing cells in many organ systems, such as the heart.

Stem cells of different origins have been observed to develop into a variety of cell types such as cardiomyocytes, hepatocytes, and epithelial cells after transplantation. Regeneration of damaged myocardial tissue has been attempted using embryonic stem cells and bone marrow-derived cells, including adult hematopoietic stem cells, mesenchymal stem cells, and bone marrow side population cells. Human CD34⁺ mononuclear cells isolated from bone marrow and peripheral blood have been successfully used for regeneration of the blood vessels after experimental MI in rats. CD34⁺ cells cocultured with rat cardiomyocytes have also been reported to differentiate into cardiomyocytes in vitro.

Sources of cells with high transdifferentiation potential or plasticity include various cell populations, such as embryonic stem cells, cord blood cells, and mesenchymal stem cells. These cell sources, however, are problematic in that they are generally not readily available and require significant manipulation. Adult human peripheral blood stem cells have been shown to differentiate into mature hepatocytes and epithelial cells of the skin and gastrointestinal tract. Unlike bone marrow derived stem cells, peripheral blood derived stem cells do not require painful extraction procedures.

### SUMMARY

The present invention generally relates to methods of improving cardiac function in a recipient host with cardiac tissue damage. More specifically, the present invention relates to methods of stimulating cardiac tissue regeneration through the introduction of peripheral blood derived stem cells into a recipient host.

In one embodiment, the present invention provides a method of stimulating heart tissue regeneration in a recipient host comprising administering peripheral blood derived stem cells to the host in an amount sufficient to result in the production of cardiomyocytes in the host's cardiac tissue.

In another embodiment, the present invention provides a method of regenerating cardiac tissue in a recipient host by obtaining peripheral blood from a donor and enriching for a peripheral blood derived stem cell based on a marker. Then an effective amount of peripheral blood derived stem cells are transplanted into the recipient host where the peripheral blood derived stem cell forms cardiomyocytes in the host's cardiac tissue. The method also may be used, in other embodiments, to treat diseased cardiac tissue.

In yet another embodiment, the present invention provides a method of obtaining a differentiated cardiac cell by obtaining a stem cell from peripheral blood and providing the stem cell to a damaged cardiac tissue in a recipient host where the stem cell differentiates into new cardiac tissue.

Both autologous and allogenic peripheral blood derived stem cells may be used in many embodiments of the present invention. In certain embodiments, autologous stem cells may be preferred.

Selected embodiments of the present invention utilize human peripheral blood derived stem cells provided to a human recipient host or human cardiac tissue. The term “peripheral blood derived stem cell” refers either to (1) a pluripotent, or lineage-uncommitted, progenitor cell, which is potentially capable of an unlimited number of mitotic divisions to either renew its line or to produce progeny cells which will differentiate into the cells that are necessary to form functional myocardial tissue (e.g., endothelial cells or endothelial-like cells, cardiomyocyte cells or cardiomyocyte-like cells, and smooth muscle cells or smooth muscle-like cells); or (2) a lineage-committed progenitor cell and its progeny, which is capable of self-renewal and is capable of differentiating into the cells necessary to form functional myocardial tissue.

The features and advantages of the present invention will be readily apparent to those skilled in the art upon a reading of the description of the embodiments that follows.

### DRAWINGS

The following figures form part of the present specification and are included to further demonstrate certain aspects of the present invention. The invention may be better understood by reference to one or more of these drawings in combination with the description of embodiments presented herein. The patent or application file contains at least one drawing executed in color. Copies of this patent or patent application publication with color drawing(s) will be provided by the Office upon request and payment of the necessary fee.

FIG. 1 shows immunofluorescence staining images of a transdifferentiated human peripheral blood CD34⁺ cell in infarcted cardiac tissue, according to an embodiment of the present invention (scale bare=10 mm);

FIG. 1A is a cluster of cells stained for human HLA-ABC;

FIG. 1B is a cluster of cells stained for cardiac troponin T;

FIG. 1C is a cluster of cells double-stained for human HLA-ABC and cardiac troponin T;

FIG. 1D is a magnification of FIG. 1C;

FIG. 1E is a single cell stained for human HLA-ABC;

FIG. 1F is a single cell stained for cardiac troponin T;

FIG. 1G a single cell double-stained for human HLA-ABC and troponin T;

FIG. 1H is a magnification of FIG. 1G;

FIG. 1I is stained for human HLA-ABC;

FIG. 1J is stained for smooth muscle-specific α-actin;

FIG. 1K is double-stained for human HLA-ABC and smooth muscle-specific α-actin;

FIG. 1L is a magnification of FIG. 1K;

FIG. 1M is stained for human HLA-ABC;

FIG. 1N is stained for VE-cadherin;

FIG. 10 is double-stained for anti-HLA-ABC and anti-VE-cadherin;

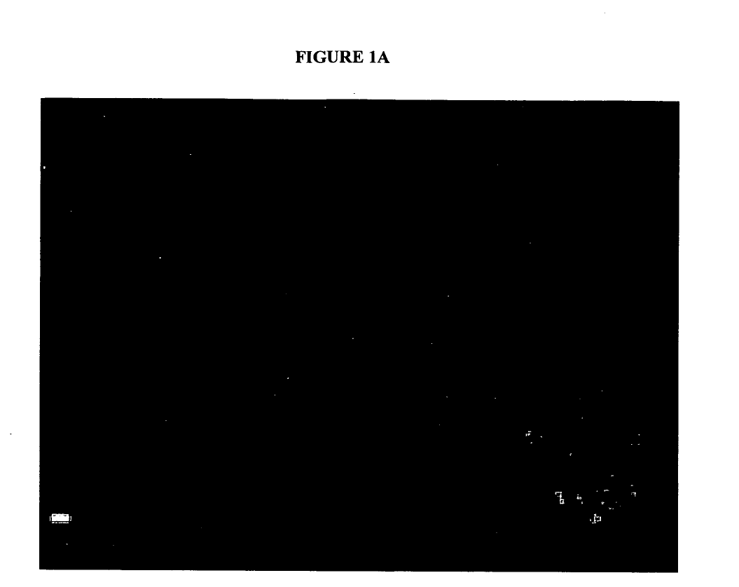

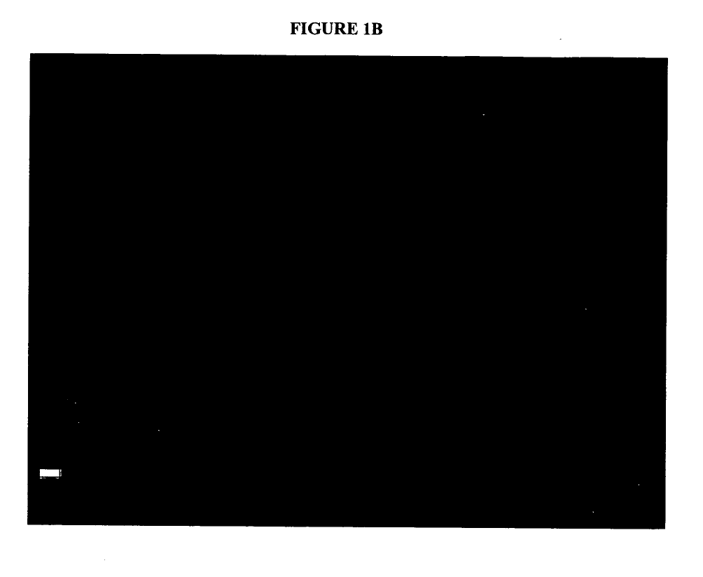

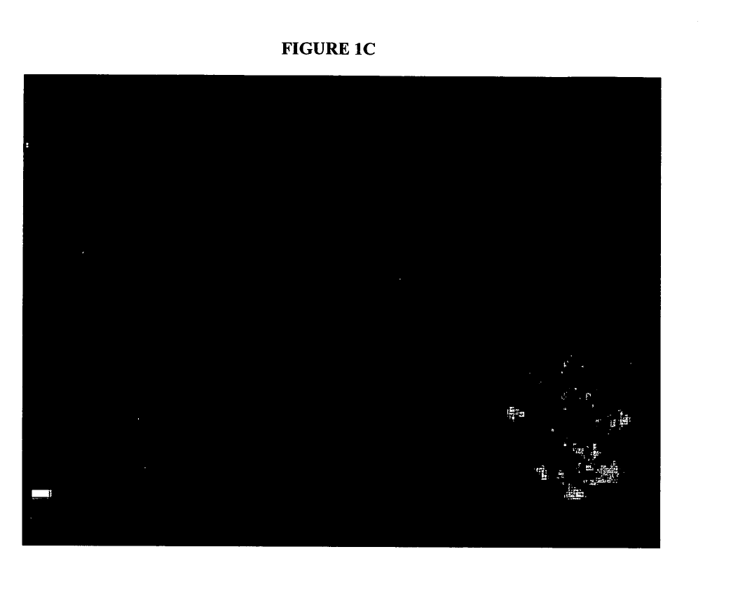

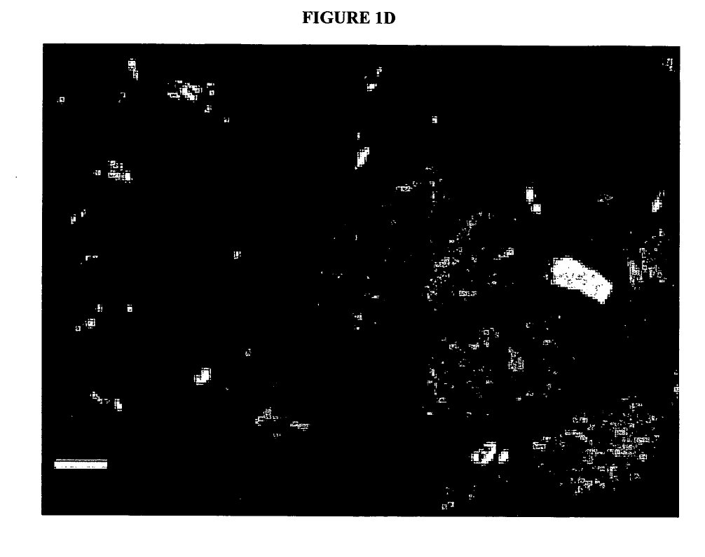

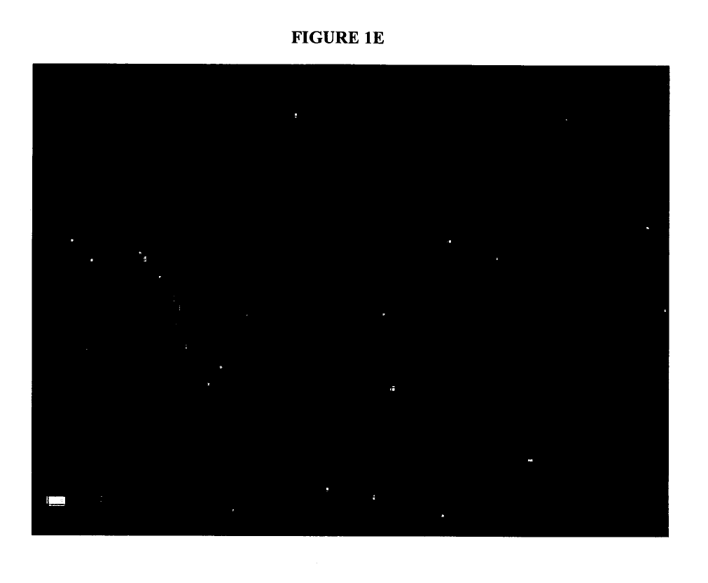

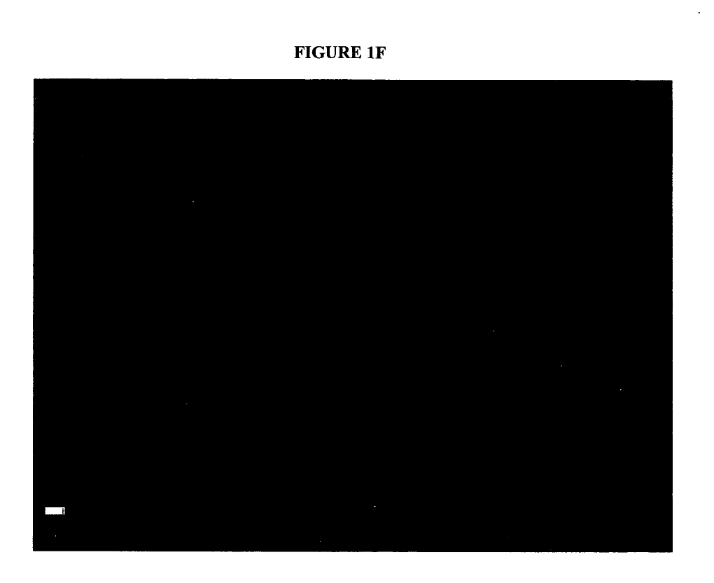

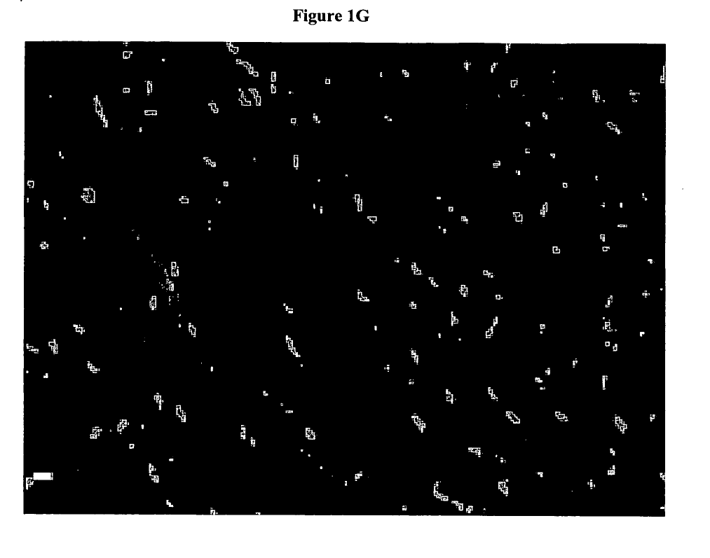

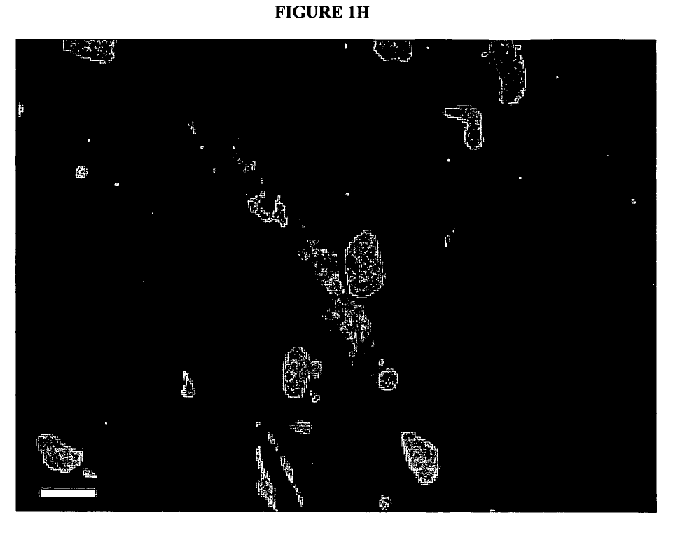

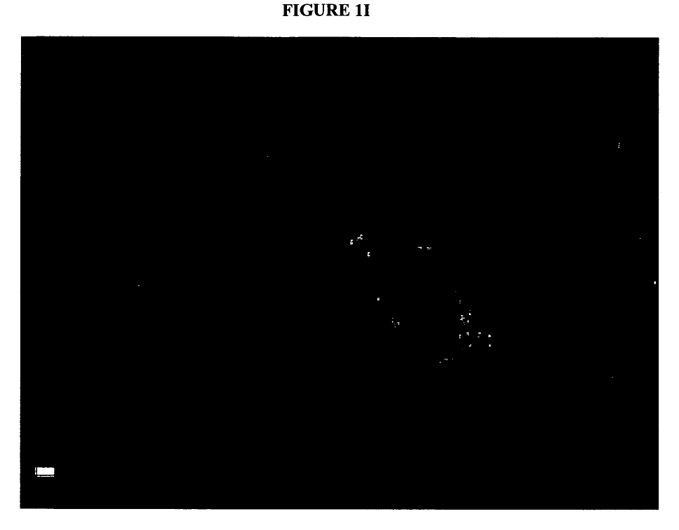

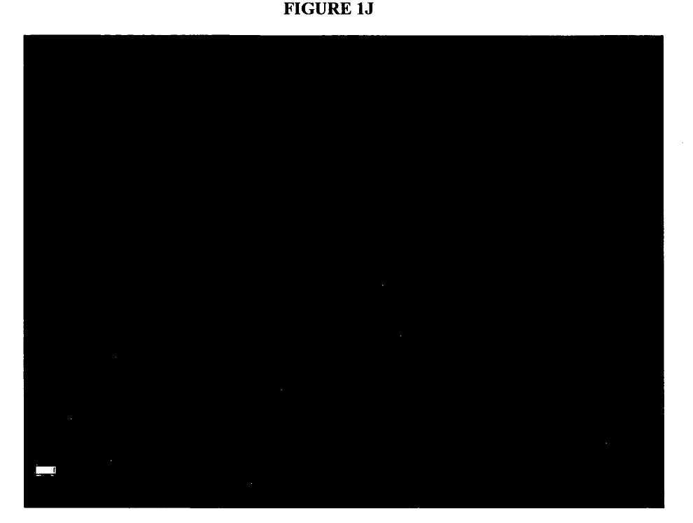

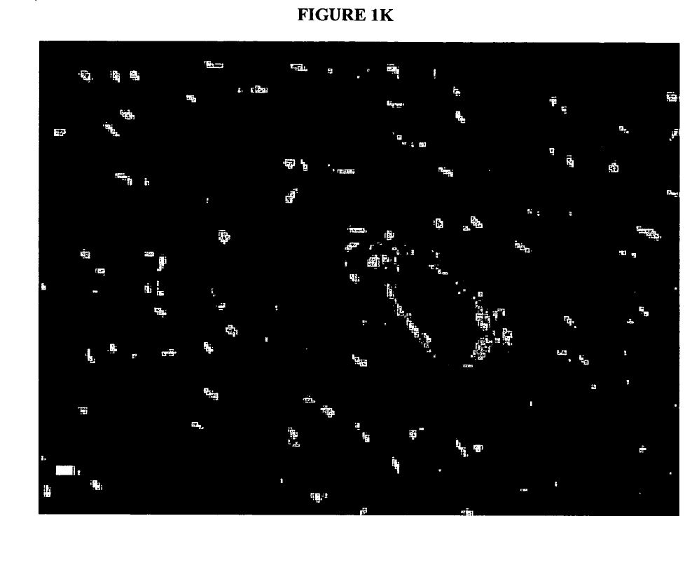

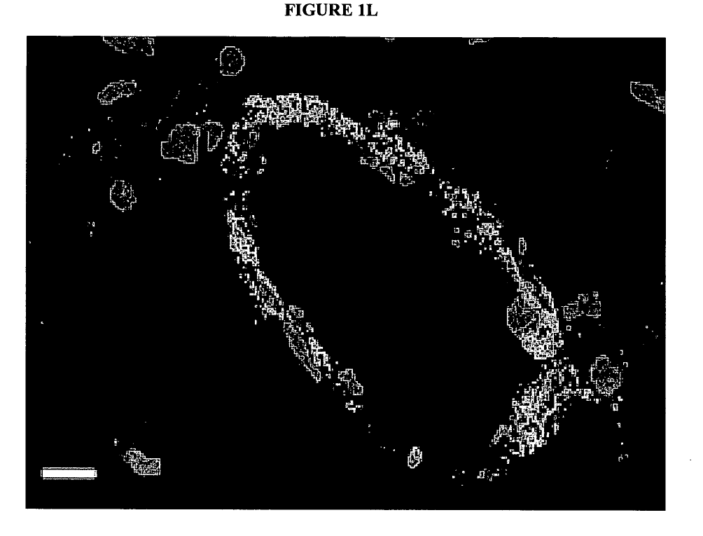

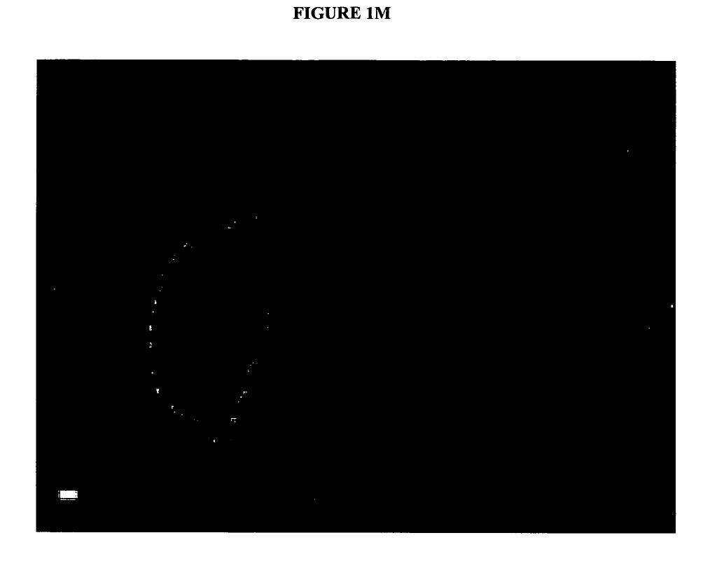

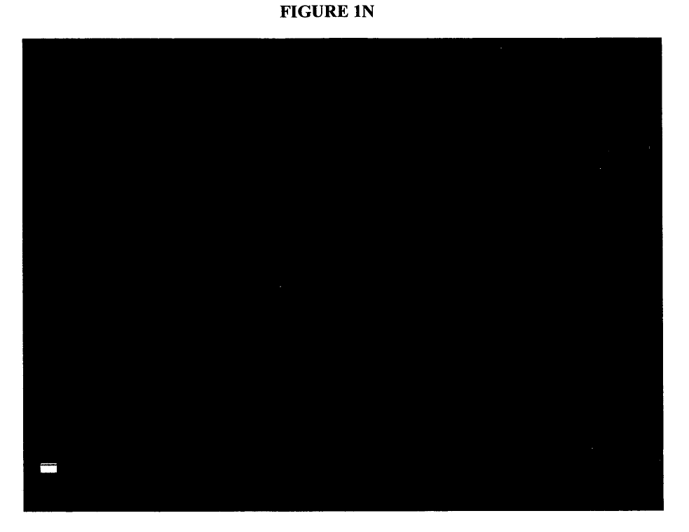

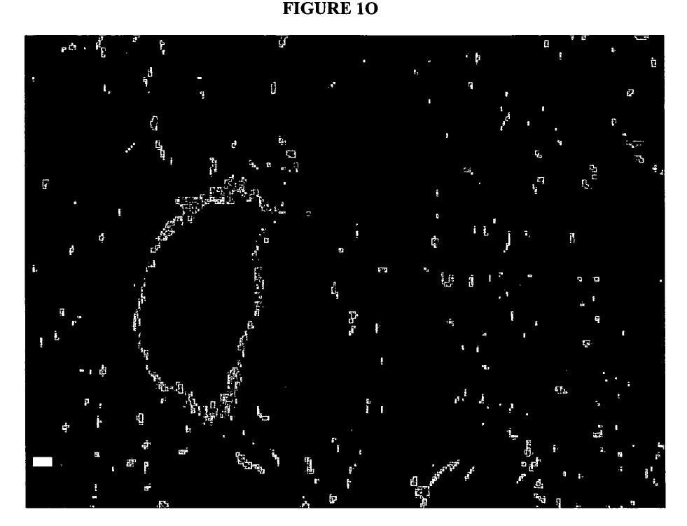

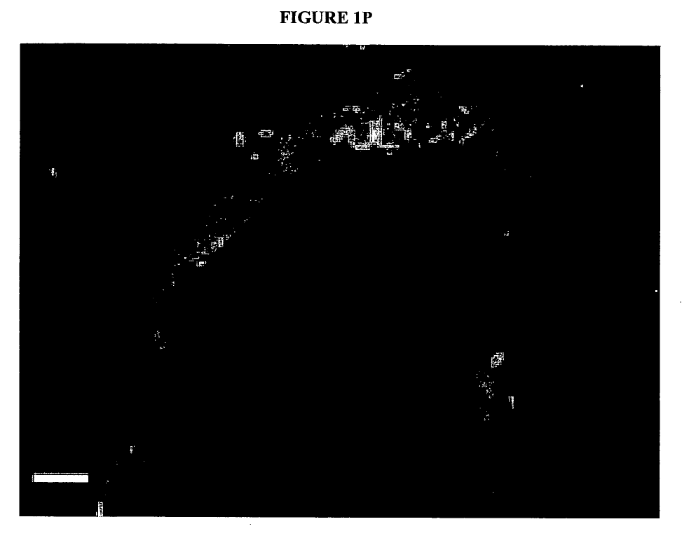

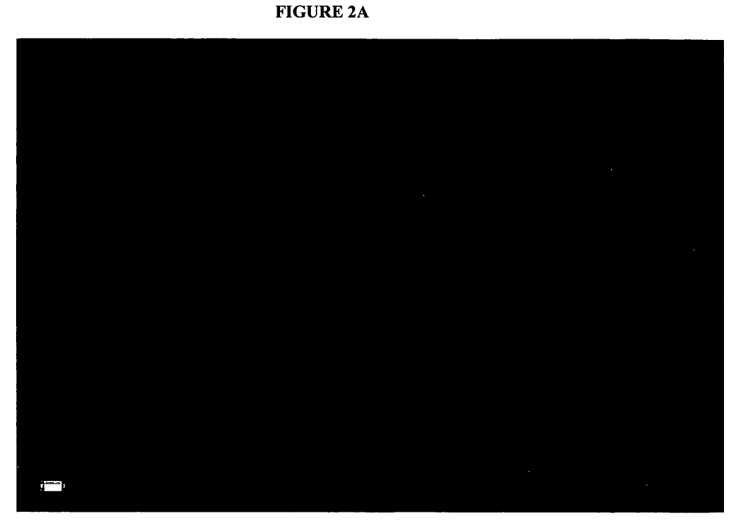

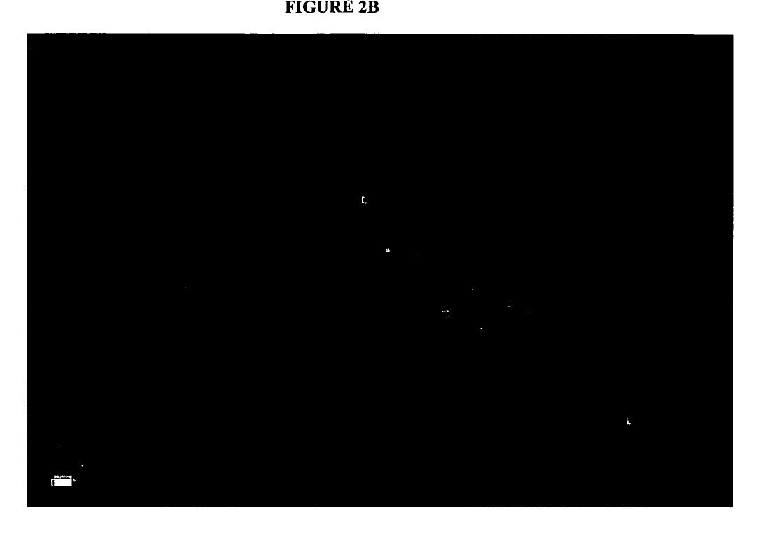

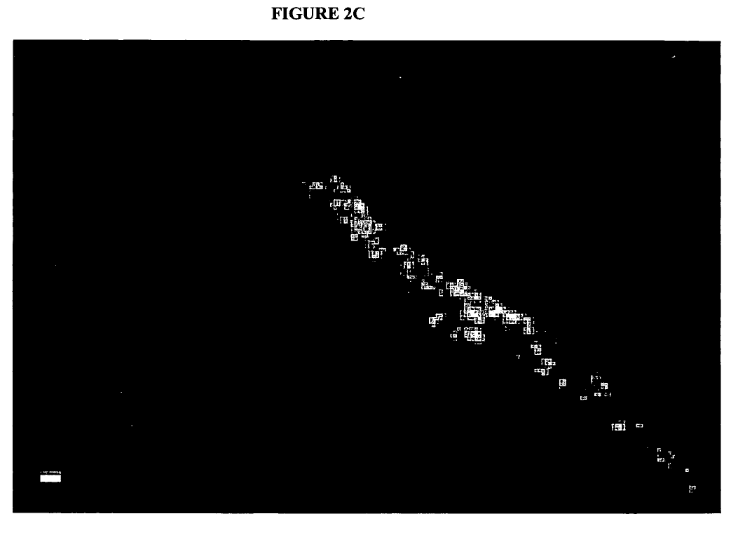

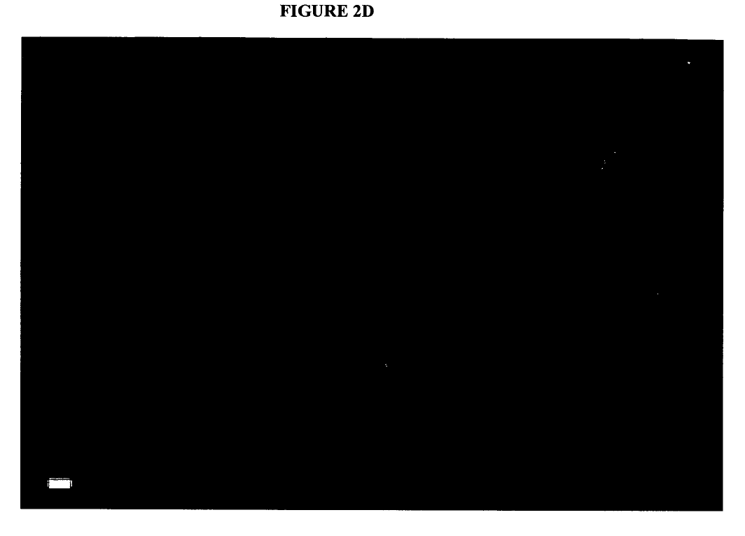

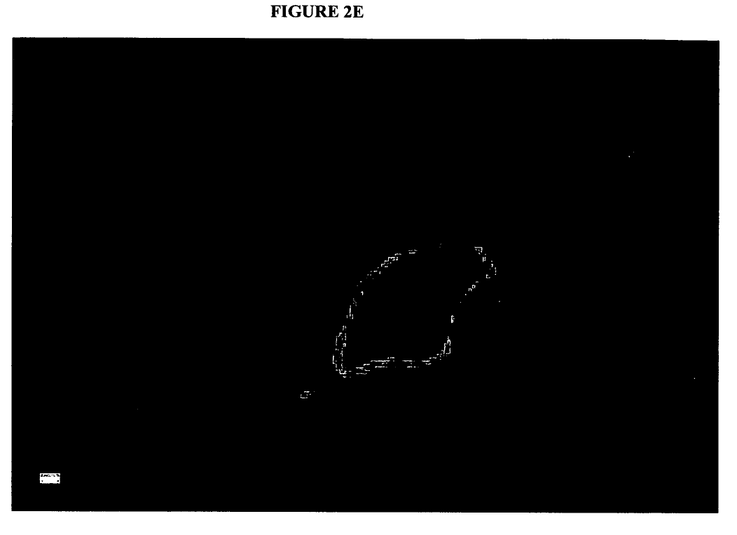

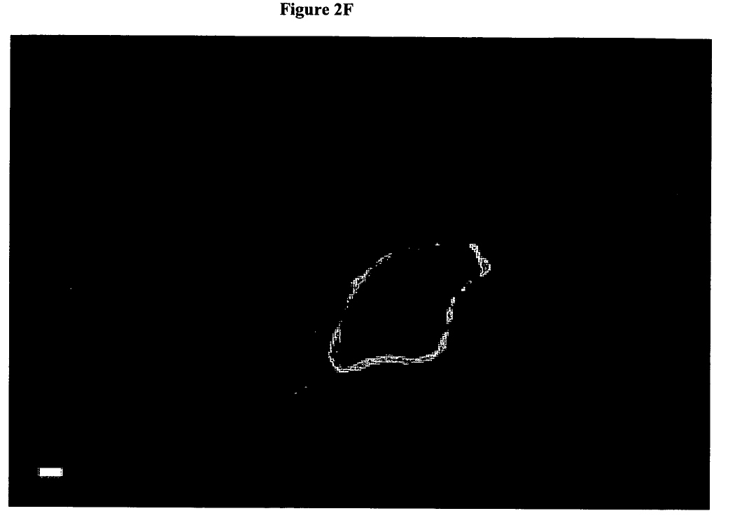

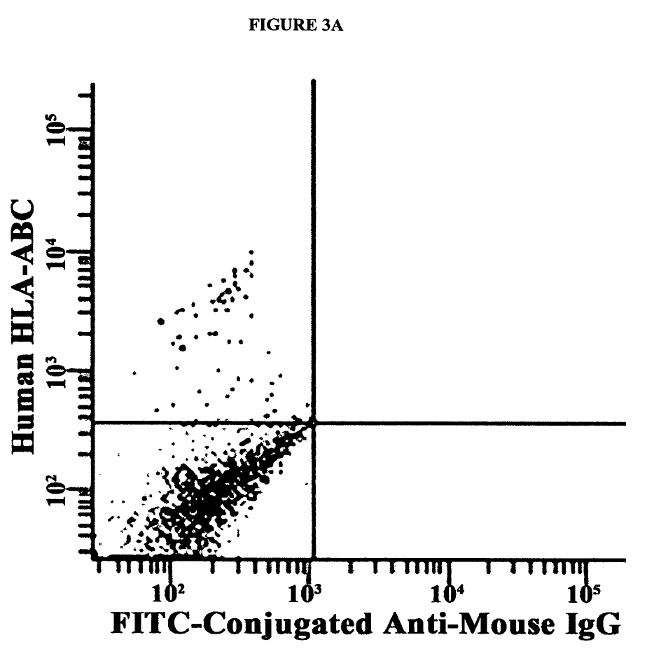

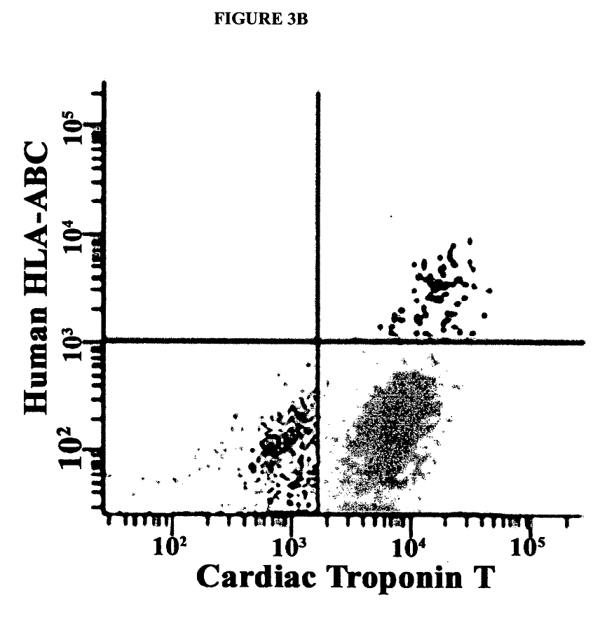

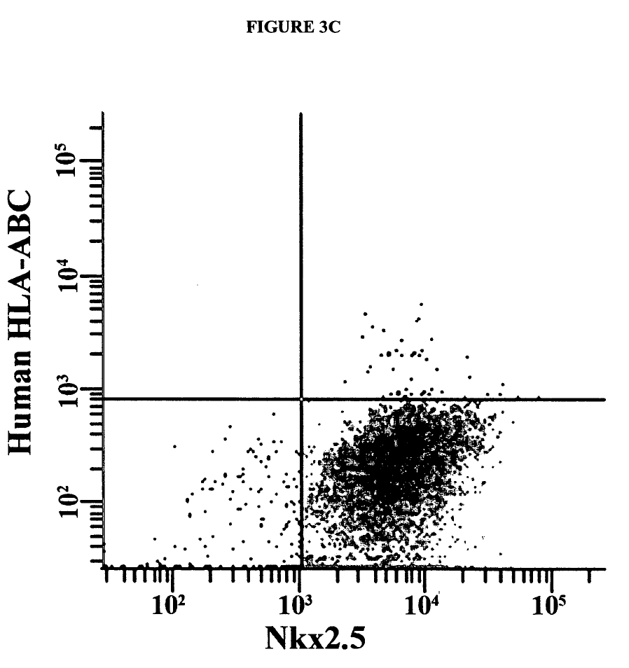

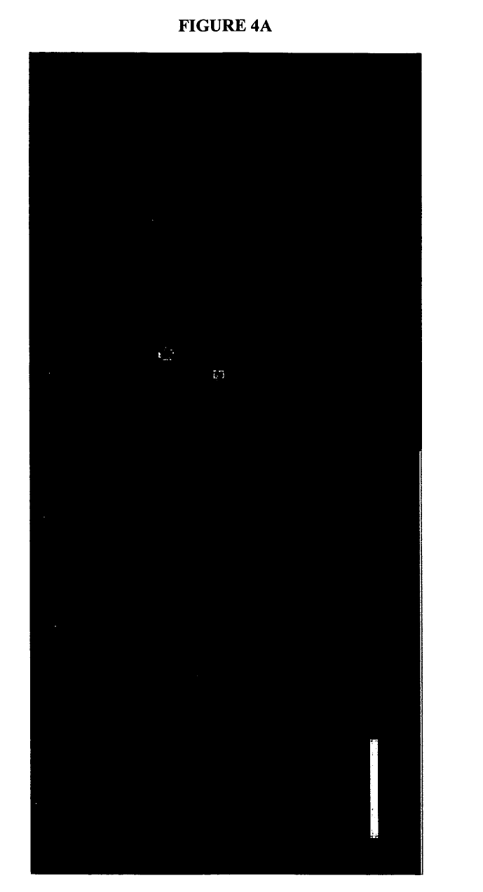

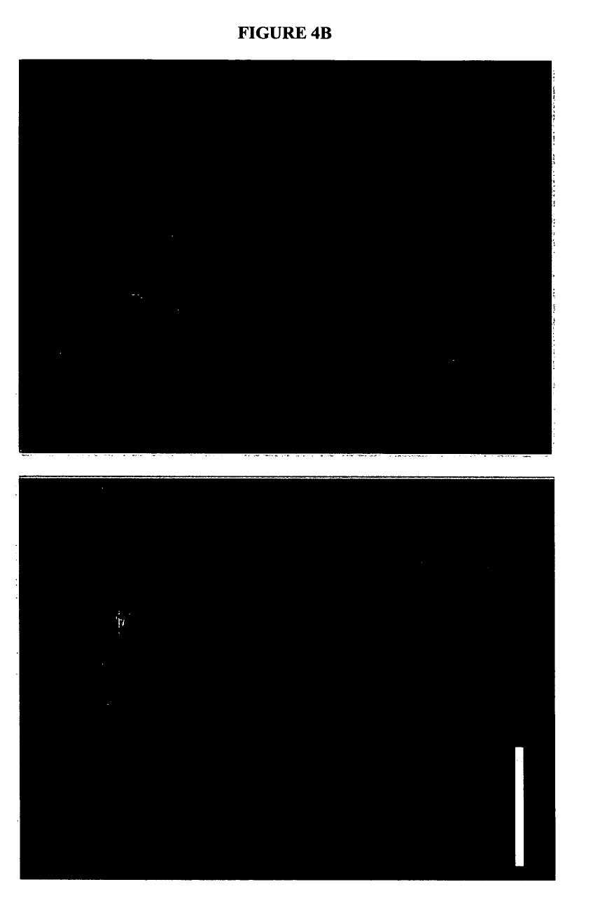

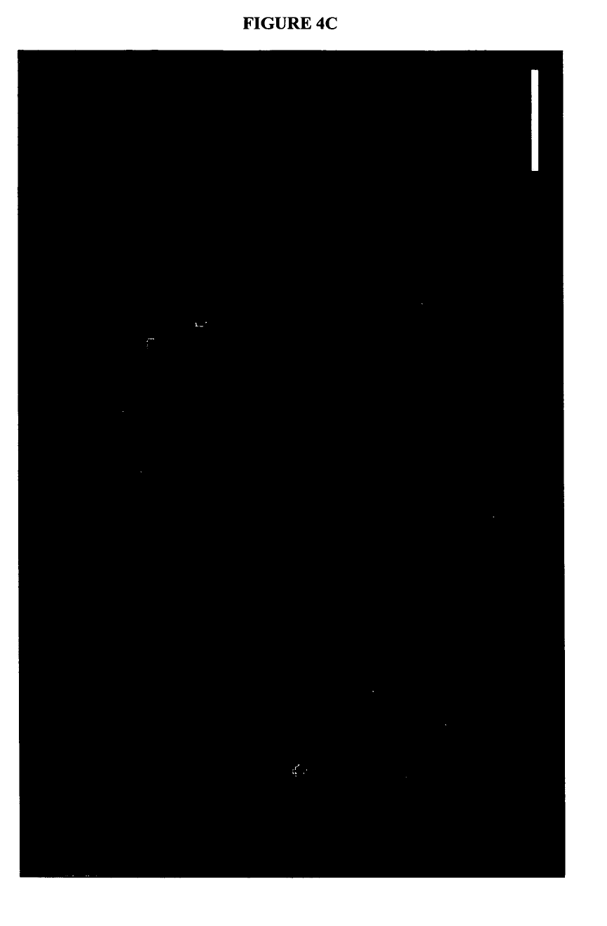

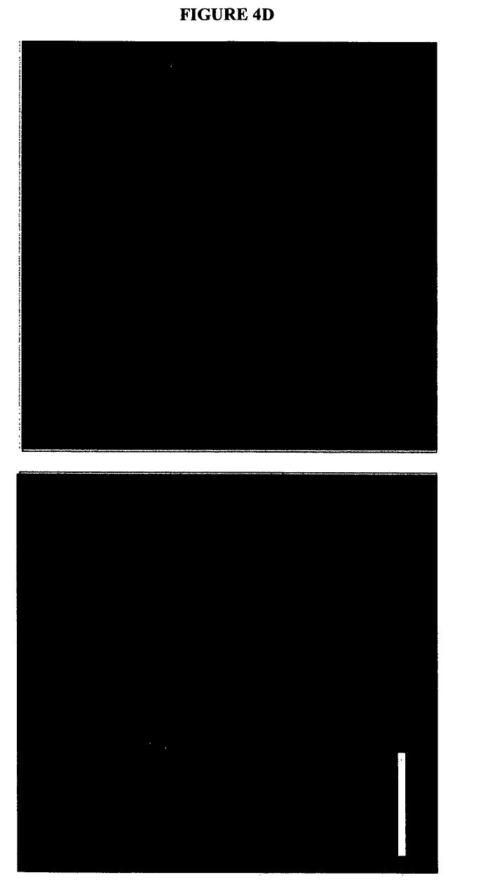

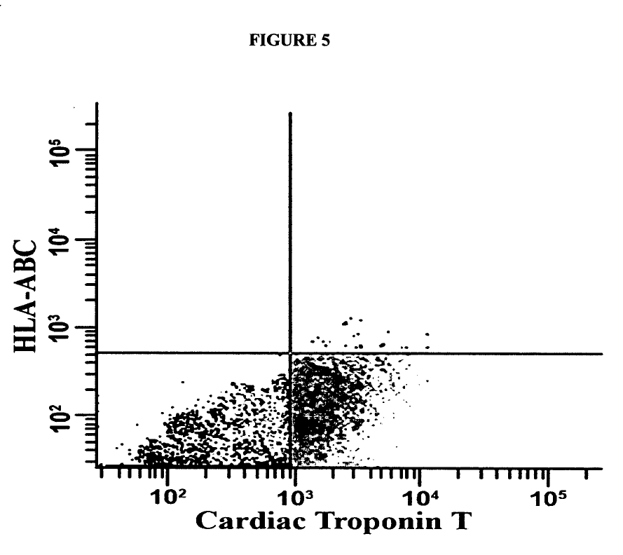

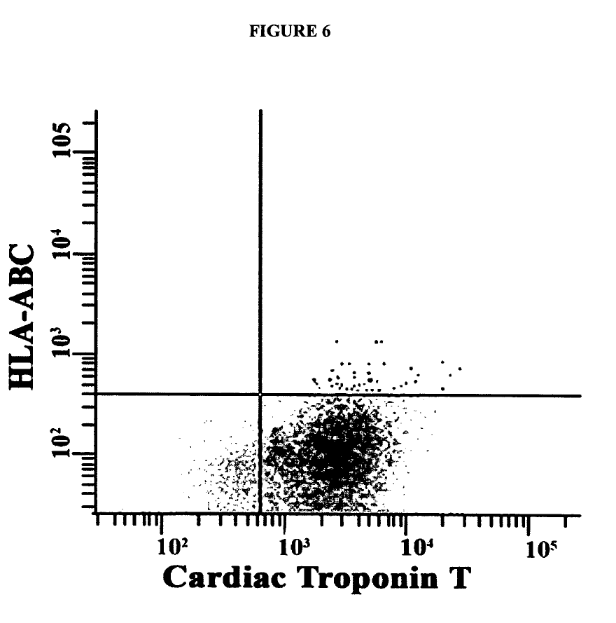

FIG. 1P is a magnification of FIG. 10;

FIG. 2 shows immunofluorescence staining images of a transdifferentiated human peripheral blood CD34⁺ cell in non-infarcted cardiac tissue (scale bare=10 mm);

FIG. 2A is stained for cardiac troponin A;

FIG. 2B is stained for human HLA-ABC;

FIG. 2C is double-stained for troponin A and human HLA-ABC;

FIG. 2D is stained for smooth muscle-specific α-actin;

FIG. 2E is stained for human HLA-ABC;

FIG. 2F is double-stained for smooth muscle-specific α-actin and human HLA-ABC;

FIG. 3 shows a flow cytometry profile of cells isolated from the heart 2 months after transplantation and stained with various antibodies;

FIG. 3A is stained with anti-human-HLA;

FIG. 3B is stained with anti-human HLA and anti-cardiac troponin T;

FIG. 3C is stained with anti-human HLA and antibody against a cardiac specific transcriptional factor, Nkx2.5;

FIG. 4 shows a FISH image of the nucleus of a Hela cell (left panel) and that of a mouse heart cell (right panel) using probes for human X chromome (red) and mouse X chromome (green) simultaneously (scale bar=5 μm);

FIG. 4A shows no cross reaction between the probes;

FIG. 4B shows 2 nuclei of HLA-negative, troponin T-positive cells with only mouse X chromosomes (green) present;

FIG. 4C shows the nuclei of about 70% HLA-positive, troponin T-positive cells with both human X chromosomes (red) and mouse X chromosomes (green) present in the adjacent two nuclei;

FIG. 4D shows the nuclei of about 30% HLA-positive, troponin T-positive cells with only human X chromosomes (red) detected;

FIG. 5 shows a flow cytometry profile of transformed cells from a mouse heart harvested 24 hours after transplantation and stained with anti-human HLA and anti-cardiac troponin T; and

FIG. 6 shows a flow cytometry profile of transformed cells from mice hearts that received transplants of mobilized human CD34⁺ stem cells and that were stained with anti-human HLA and anti-cardiac troponin T.

### DESCRIPTION

The present invention generally relates to methods of improving cardiac function in a recipient host with cardiac tissue damage. More specifically, the present invention relates to methods of stimulating cardiac tissue regeneration through the introduction of peripheral blood derived stem cells into a recipient host.

The present invention is based in the observation that peripheral blood derived stem cells may transdifferentiate into non-hematopoietic tissues and such transdifferentiation is augmented by local tissue injury. Beneficial cell fusion of the stem cell and cardiac cells may also occur. The present invention may use a subset of the peripheral blood derived stem cell population, such as the CD34⁺ set. The invention includes methods of using peripheral blood derived stem cells to regenerate myocardial tissue in a recipient host. It may also include various systems, compositions, and cells or tissues related to these methods.

In certain embodiments of the present invention, peripheral blood derived stem cells are administered to a recipient host in need of new myocardial tissue. The peripheral blood derived stem cells may be introduced into the recipient's heart, where the peripheral blood derived stem cells may undergo proliferation and transdifferentiation and possibly also cell fusion. This leads to the production of new myocardial tissue that includes cardiomyocytes, endothelial cells, and smooth muscle cells. In other embodiments, the present invention provides a method of promoting differentiation of peripheral blood derived stem cells into cardiomyocytes, endothelial cells, and/or smooth muscle cells. The method includes obtaining a peripheral blood derived stem cell with cardiomyocyte, endothelial, and/or smooth muscle cell potential from a donor, such as an autologous donor, and providing the stem cell to damaged cardiac tissue. In certain exemplary embodiments, the present invention provides for the transdifferentiation of human CD34⁺ peripheral blood derived stem cells into cardiomyocytes.

Peripheral blood derived stem cells may be obtained from a variety of different donor sources. In a certain embodiment, autologous peripheral blood derived stem cells are obtained from the recipient host who is to receive the peripheral blood derived stem cells. This approach is especially advantageous because the immunological rejection of foreign tissue is avoided. In other embodiments, allogenic peripheral blood derived stem cells may be obtained from donors who are genetically compatible with the recipient and share the same transplantation antigens on the surface of their blood cells. For example, a donor who would be suitable for a heart transplant would likely also be a suitable peripheral blood derived stem cell donor. In general, human stem cells are preferred. Peripheral blood derived stem cells may be obtained from the donor using standard phlebotomy or apheresis techniques.

Suitable peripheral blood derived stem cells may be detected, enriched, and/or purified using cell surface markers. A “marker” is any of the various surface antigens present on a stem cell. A molecule is a marker of a desired cell type if it is found on a sufficiently high percentage of cells of the desired cell type, and found on a sufficiently low percentage of cells of an undesired cell type, that one may achieve a desired level of purification of the desired cell type from a population of cells having both desired and undesired cell types by selecting for cells in the population of cells that have the marker. Examples of suitable markers characteristic of a stem cell include, but are not limited to, CD144, CD202b, P1H12, AC133, CD34, and c-kit.

Before administration into the recipient, peripheral blood derived stem cells may be isolated or enriched, individually or in populations. The term “enriched” refers to cells that are present in numbers greater than normally occurring. The term “isolated” or “purified” refers to cells that are substantially free of cells that are carrying markers associated with lineage dedication. Peripheral blood derived stem cells may be isolated or enriched using any of the various markers expressed, or not expressed, on certain stem cells in combination with suitable separation techniques. Cells may be selected based on the presence or absence of more than one marker. Suitable separation techniques include, but are not limited to, immunomagnetic bead separation, affinity chromatography, and fluorescence activated cell sorting (FACS), density gradient centrifugation, and flow cytometry.

The peripheral blood derived stem cells may be administered to the recipient host in an amount effective to achieve its intended purpose. More specifically, an amount effective may mean an amount sufficient to lead to the development of new cardiac tissue and restoration of cardiac function, thereby alleviating the symptoms associated with cardiac dysfunction.

The number of peripheral blood derived stem cells needed to achieve the purposes of the present invention will vary depending on, among other things, the degree of cardiac tissue damage. For example, the stem cells are administered in an amount effective to restore cardiac function. The dose range of stem cells to be used in the practice of the invention may vary between from about 10⁷ to about 10⁸ cells. In some cases it may be necessary to use dosages outside this range and/or to use multiple or repeat dosages or treatments, as will be apparent to those of skill in the art.

Determination of effective amounts is within the capability of those skilled in the art. The effective dose may be determined by using a variety of different assays and tests that are designed to detect restoration of cardiac function, including, but not limited to, magnetic resonance imaging, positron emission tomography, and echocardiogram.

The stem cells may be administered to the recipient host in one or more physiologically acceptable carriers. Carriers for these cells may include, but are not limited to, solutions of phosphate buffered saline containing a mixture of salts in physiologic concentrations or Ring's lactate.

In certain embodiments of the present invention, the stem cells may be delivered to injured tissue. Tissue injury may enhance transdifferentiation into myocardial tissue. The isolated or enriched peripheral blood derived stem cells may be administered by injection or by alternative delivery methods into the center, bordering zone, or neighboring areas of an ischemic tissue, e.g., the myocardium, coronary blood vessels, or peripheral blood vessels. In one aspect, the stem cells are delivered to underperfused tissue such as tissue found in chronic ischemia. Such tissue includes, but is not limited to, ischemic tissues, cardiac muscle tissues, vascular tissues, or combinations thereof. In another aspect, the stem cells may be introduced to an area of tissue near or within a distance sufficient to enable the transplanted cells to migrate to the ischemic tissue. In general, delivery of the stem cells may be accomplished with the use of any medical device or technique for delivery of cells that is known, with the benefit of this disclosure, to those skilled in the art. Cells may be administered in any manner sufficient to allow them access to the heart. Methods may rage from injection into the general circulation to direct cardiac injection, such as via catheter. In some instances, it may be necessary to administer the stem cells more than once to restore cardiac function.

Recipient hosts suitable to receive stem cells as described in the present invention include any hosts with cardiac damage. Such damage may be due to infarction and other acute events, but also may be due to chronic problems. Further, such injury may be drug induced, for example by chemotherapy, or the result of infection.

To facilitate a better understanding of the present invention, the following examples of specific embodiments are given. In no way should the following examples be read to limit or define the entire scope of the invention.

### EXAMPLES

### Example 1

Female SCID mice (CB-17) weighing 16 to 23 grams were used (Charles River Laboratories, Wilmington, Mass.) to demonstrate the transdifferentiation of human peripheral blood derived CD34⁺ stem cells in vivo.

For comparison, three groups of mice were tested. The first group of mice had about 2.5 million human peripheral blood derived CD34⁺ stem cells injected into the left ventricle without surgical manipulation, and the heart was harvested at 24 hours, 4 days, 12 days, 30 days, or 60 days after stem cell transplantation. The second group of mice received experimentally induced MI followed by injection of about 2.5 million human peripheral blood derived CD34⁺ stem cells through the tail vein 16 hours later and where then harvested 60 days post transplantation. A third group of mice received the same treatment except that a sham surgery was preformed.

The peripheral blood derived CD34⁺ stem cells were obtained by fractionation of human peripheral blood using immunomagnetic beads as described in A. Ferrajoli, et al., Stem Cells, 11:112-19 (1993), the relevant disclosure of which is incorporated herein by reference.

The second group of mice underwent a left anterior descending ligation procedure: the mice were ventilated with 100% oxygen using a rodent ventilator (Inspira ASV, Harvard Apparatus, Inc.); the chest was opened; a 7-0 silk suture (Ethicon, Inc., Johnson & Johnson Co.) was passed with a tapered needle under the left anterior descending coronary artery 1 to 2 millimeters from the tip of the left atrium; and the 2 ends of the suture were tied to induce MI. The third group of mice underwent the same procedure except for the left anterior descending ligation.

Heart tissue was recovered and analyzed using immunofluorescence staining. The mouse hearts from the three groups of mice were recovered and prepared as follows. Hearts were removed, embedded in OCT, snap frozen in liquid nitrogen, and stored at −80° C. Mouse hearts in OCT blocks were sectioned, and 5 micrometer serial sections were collected on slides followed by fixation with 3.7% paraformaldehyde (pH 7.4) at 4° C. for 5 minutes and immediately stained.

The recovered and prepared heart samples were analyzed by immunofluorescence as follows. After rinsing the slides with phosphate buffered saline (PBS) 3 times, the slides were blocked at room temperature for 30 minutes in PBS containing 5% horse serum and incubated with primary antibodies at room temperature for 1 hour. The slides were rinsed 3 times and incubated with the secondary antibody at room temperature for 30 minutes. Paired primary and secondary antibodies were used for double staining. The slides were rinsed again, and DAPI solution was applied for 5 minutes. Reagents used were anti-human leukocyte antigen (HLA)-ABC (BD Biosciences); anti-troponin T antibody (Santa Cruz Biotechnology) reacts against cardiomyocytes of human and mouse; anti-α-smooth muscle actin (Spring Biosciences) reacts against both human and mouse smooth muscle actin; and anti-VE-cadherin reacts against endothelial cells (Bender Medsystem). Secondary antibodies were Alexa Fluor 488-conjugated goat anti-mouse IgG (Molecular Probes) for anti-HLA and goat anti-rabbit IgG Rhodamine (Santa Cruz Biotechnology) for anti-smooth muscle actin and anti-VE-cadherin antibodies.

In the mice of group two, which sustained MI for 60 days, clusters of cells (FIGS. 1A-1D) and single cells (FIGS. 1E-1H) with the morphological appearance typical of cardiomyocytes were stained positively with both anti-human HLA-ABC and anti-cardiac troponin T in the peri-infarct area. But, no cardiomyocytes derived from human peripheral blood derived CD34⁺ stem cells were observed in the infarct zone. Moreover, no transdifferentiated cardiomyocytes were found in tissue sections of group three mice (sham surgery) after cell transplantation.

Blood vessels that reacted with the anti-HLA antibody were seen mainly in the infarct area of group two mice. Double staining for HLA and smooth muscle α-actin indicated that human CD34-derived smooth muscle cells had participated in the neovascularization after acute MI in the group two mice (FIGS. 1I-1L). Double staining of the blood vessels with anti-HLA and anti-VE-cadherin confirmed that human CD34⁺ stem cells transdifferentiated into mature endothelial cells (FIGS. 1M-1P).

Group one mice, without MI, demonstrated no transdifferentiation at 24 hours, 4 days, 30 days, and 60 days after injection. Some group one mice (1 out of 7, 12 days post injection) did show cardiomyocytes and blood vessels after double staining with anti-HLA and cardiac troponin (FIGS. 2A-2C) or smooth muscle α-actin (FIGS. 2D-2F). Hence, the observation that the transdifferentiation potential of human peripheral blood derived CD34⁺ stem cells is augmented by local tissue injury.

The above example demonstrates, among other things, that the methods of the present invention are suitable for use in a recipient host with cardiac tissue damage.

### Example 2

Female SCID mice (C3H, Jackson Laboratory, Bar Harbor, Me.) weighing 14-18 grams were used study the transformation mechanism of peripheral blood derived stem cells. Experimental MI was induced as described above. Two million human peripheral blood derived CD34⁺ stem cells, obtained as described above, were injected into the left ventricles of 7 mice 30-40 minutes after MI was induced. Three control mice received the same MI procedure, but no cell transplantation.

The hearts were harvested 60 days after cell transplantation or MI (control animals). Heart cells were isolated as described in E. K. Hudson, et al., Am J. Physiol., 271:H422-27 (1996) (the relevant disclosure of which is incorporated herein by reference), with the following modifications. The heart was cut into 6 pieces and placed in a 10 milliliter beaker containing 2 milliliters of the enzymatic solution, nominally calcium-free Hepes-buffered salt solution (ADS buffer) containing elastase (0.3 mg/mL, Worthington Biochemical, Freehold N.J.), type II collagenase (0.21 mg/mL, Worthington Biochemical, Freehold N.J.), and pancreatin (0.6 mg/mL, Sigma, MO). The tissues were then incubated repeatedly at 37° C. for 15 minutes each time. The isolated cells were filtered through a cell strainer with a pore size of 70 micrometers and fixed (20 min. at 4° C.) with Cytofix/Cytoperm kit (BD Pharmingen), stored at 4° C. overnight before FACS analysis.

The filtered cells were permeabilized using the Cytoperm/wash kit (BD Pharmingen) for 20 minutes at 4° C. and incubated with a monoclonal antibody against cardiac troponin T (1:200, clone 1A11, Advanced Immunochemical, Long Beach, Calif.), or incubated with non-specific mouse IgG 2b for 30 minutes at 4° C. After 3 washes, cells were incubated with goat-anti mouse IgG (1:1000) conjugated with Alexa Fluor 488 (Molecular Probes, Eugene, Oreg.) for 30 minutes at 4° C. In a separate set of experiments, mouse heart cells were incubated with a polyclonal antibody (goat, Santa Cruz Biotechnology, Santa Cruz, Calif.) against a cardiac specific transcription factor, Nk×2.5 or with non-specific goat IgG for 30 minutes at 4° C. Cells were washed and incubated with chicken-anti goat IgG (1:1000) conjugated with Alexa Fluor 488 for 30 minutes at 4° C. After 3 washes, cells incubated with anti-cardiac troponin or with anti-Nkx2.5 were incubated again with PE-conjugated anti-human HLA-ABC (clone W6/32, Cedarlane Laboratories, Homby, Ontario Canada) or PE-conjugated mouse IgG 2a for 30 min. For endothelial cell detection, isolated cells were incubated with anti-VE-cadherin (1:100, Bender MedSystems, Calif.), then with the secondary antibody conjugated with Alexa Fluor 488. Cells were analyzed and sorted on a FACS Aria flowcytometer (BD Biosciences). Gates were established by nonspecific Ig binding in each experiment. Approximately 30-40% of the entire population was sorted for the double positive cells.

Sorted cells were analyzed using PCR. Genomic DNA was isolated from heart cells sorted by expression of cardiactroponin T or by dual expression of human HLA-ABC and cardiac troponin T, and was used to detect HLA expression by PCR. PCR analysis was performed using the primers derived from HLA-B Exon 5-7 with advantage cDNA PCR kit (Clontech). The following touchdown PCR program was used: 3 min at 94° C.; 5 cycles of 30 seconds each at 94° C., 40 seconds each at 66° C. and 50 seconds each at 72° C.; 30 cycles of 30 seconds each at 94° C., 40 seconds each at 64° C. and 50 seconds each at 72° C.; and 1 cycle of 5 minutes at 72° C. The PCR products were analyzed by electrophoresis of 1% agarose gel and the size of PCR amplicon derived from HLA-B gene was approximately 280 base pairs.

Interphase fluorescence in situ hybridization (FISH) was used to further analyze the cells. The collected double positive cells were spun onto a slide and fixed immediately with 3:1 methanol:acetic acid solution for 30 minutes. In order to quench the residual fluorescence from cell sorting, the slides were exposed to white light for 120 hours at 4° C. Complete diminishment of the residual fluorescence was confirmed by examination under an epifluorescence microscope (Nikon Eclipse TE-2000-U). Slides were briefly fixed in 3:1 methanol:acetic acid again and were pre-denatured, dehydrated, and denatured according to the manufacturer's protocol. Slides were hybridized with a FITC-conjugated DNA probe for mouse X chromosomes (ID Labs, London, ON Canada) and a PE-conjugated probe for human X chromosomes (Qbiogene, Carlsbad, Calif.) overnight at 37° C. in a humidified chamber. After post-hybridization wash, slides were counterstained with DAPI (0.02 μg/ml) and examined using an epi-fluorescence microscope (Nikon Eclipse TE 2000-U).

In order to evaluate engraftment of the peripheral blood derived CD34⁺ stem cells in the heart and transformation of these cells into the cardiomyocytes, we examined isolated cells by FACS analysis using specific antibodies against HLA-ABC, a surface marker for human cells, and cardiac troponin T, a cardiomyocyte-specific marker. HLA-positive cells were detected in all 4 mice examined. Approximately 2% (2.0±0.4%) of the total cells from the heart were human HLA-positive (FIG. 3A), whereas cells from control mice with induced MI, but not injected with CD34+ stem cells, were all HLA-negative. FACS analysis of heart cells double-stained with antibodies against HLA and cardiac troponin T (FIG. 3B) and with antibodies against HLA and Nkx2.5 (FIG. 3C) demonstrated that about 1% of cells were double positive, suggesting that these cardiomyocytes originated from the transplanted human cells. To ensure that immuno-staining accurately reflects the genotype of isolated cells, DNA from the sorted double-* positive cells and the cells stained only with anti-cardiac troponin was extracted for PCR detection of human HLA-B gene. HLA-B fragment was only amplified in double-positive cells. These results show that the double positive cells are of human origin.

The population of double-positive cells was collected by cell sorting and examined by using FISH analysis in which specific probes for human and mouse X chromosomes were used simultaneously. The specificity of the probes was tested in mouse heart cells and human Hela cells by incubating these cells with both probes, and we confirmed that these 2 probes did not cross-react (FIG. 4A). In the nuclei derived from cells that were troponin T-positive, but HLA-negative, only mouse X chromosomes were detected (FIG. 4B). Since the recipient mice were female, two X chromosomes were observed in each nucleus. In troponin T-positive and HLA-positive cells, about 70% of the nuclei contained both human and mouse X chromosomes (Table 1 and FIG. 4C), suggesting that cell fusion had occurred. Since the human donor is male, one human X chromosome was paired with two mouse X chromosomes in each nucleus (FIG. 4C). However, about 30% of the nuclei of troponin T-positive, HLA-positive cells contained only human X chromosomes (Table 1 and FIG. 4B), suggesting that transdifferentiation of peripheral blood derived CD34⁺ stem cells have also taken place. Analysis was also made for HLA and Nkx-2.5 sorted cells in three mice and similar findings were obtained (Table 1). On the other hand, only human X chromosomes were detected in about 97% of cells stained positive to both anti-HLA-ABC and anti-VE-cadherin (Table 2). Together, Tables 1 and 2 show that cell fusion tends to be the predominant, but not exclusive method by which new cardriomyocytes are formed. In contrast, new endothelial cells are formed almost entirely by transdifferentiation.

TABLE 1

| | X Chromosome in Nuclei | | | Total |
|---|---|---|---|---|
| Double-positive | Human and | | | Nuclei |
| cells | Mouse | Human | Mouse | Counted |
| HLA⁺ and | 70 (70%) | 28 (28%) | 2 (2.0%) | 100 |
| Troponin⁺, n (%) | | | | |
| HLA⁺ and | 84 (71.2%) | 31 (26.3%) | 3 (2.5%) | 118 |
| Troponin⁺, n (%) | | | | |
| HLA⁺ and | 96 (78.7%) | 24 (19.7%) | 2 (1.6%) | 122 |
| Troponin⁺, n (%) | | | | |
| HLA⁺ and | 91 (71.1%) | 36 (28.1%) | 1 (0.8%) | 128 |
| Troponin⁺, n (%) | | | | |
| HLA⁺ and Nkx2.5⁺, | 91 (78.4%) | 19 (16.4%) | 6 (5.2%) | 116 |
| n (%) | | | | |
| HLA⁺ and Nkx2.5⁺, | 47 (69.1%) | 17 (25%) | 4 (5.9%) | 68 |
| n (%) | | | | |
| HLA⁺ and Nkx2.5⁺, | 86 (74.8%) | 26 (22.6%) | 3 (2.6%) | 115 |
| n (%) | | | | |
| Mean ± SE | 73.3 ± 1.5% | 23.7 ± 1.7% | 2.9 ± 0.7% | |

TABLE 2

| | X Chromosome in Nuclei | | | Total |
|---|---|---|---|---|
| HLA⁺ and VE- | Human and | | | Nuclei |
| Cadherin⁺ | Mouse | Human | Mouse | Counted |
| Mouse 1, n (%) | 1 (1%) | 101 (97.1%) | 2 (1.9%) | 104 |
| Mouse 2, n (%) | 0 (0%) | 127 (97.7%) | 3 (2.3%) | 130 |
| Mouse 3, n (%) | 1 (0.8%) | 119 (98.4%) | 1 (0.8%) | 121 |
| Mouse 4, n (%) | 0 (0%) | 136 (99.3%) | 1 (0.7%) | 137 |
| Mouse 5, n (%) | 5 (3.6%) | 131 (95%) | 2 (1.4%) | 138 |
| Mouse 6, n (%) | 3 (2%) | 141 (96%) | 3 (2%) | 147 |
| Mean ± SE | 1.2 ± 0.6% | 97.3 ± 0.6% | 1.5 ± 0.2% | |

Heart cells harvested at different time points also were analyzed. Using the same approach described above, HLA-positive and HLA/cardiac troponin T-double positive cells appeared in the heart as early as 24 hours after transplantation (FIG. 5). Furthermore, 6 months after transplantation both HLA-positive and HLA/cardiac troponin T-positive cells existed in the heart, and after a year HLA-positive and HLA/cardiac troponin T-positive cells could still be detected. Hence, the observation that both cell fusion and transdifferentiation may be involved in the transformation of transplanted human peripheral blood stem cells into cardiomyocytes in injured hearts.

The above example demonstrates, among other things, that the methods of the present invention are suitable for use in a recipient host with cardiac tissue damage.

### Example 3

G-CSF-mobilized human peripheral blood derived CD34⁺ stem cells (2×10⁶/mouse, intra-ventricular injection) were transformed into SCID mice 1 hour after experimental MI. Analysis of the mobilized human peripheral blood derived CD34⁺ stem cells by FACS showed that the population contained at least 97% of human peripheral blood derived CD34⁺ stem cells, and 86% of these cells were CD133⁺.

Mouse heart was removed and cells isolated 24 hours after transplantation and analyzed using FACS. Cells doubly stained positive with anti-HLA-ABC and anti-cardiac troponin T were isolated using FACS and used for FISH detection of human and mouse chromosomes. The transplanted cells were able to home to the heart and transform into cardiomyocytes at 24 hours after transplantation (FIG. 6). Moreover, the frequencies of total HLA-positive cells and the donor-derived cardiomyocytes (HLA and cardiac troponin T double positive) were not different when compared with previously used human peripheral blood derived CD34⁺ stem cells analyzed at the same time point. There was no difference in the percentage of nuclei containing both human and mouse X chromosomes as the marker of cell fusion between 2 strains of mice. Hence, the observation that different preparations of human peripheral blood stem cells can transform into cardiomyocytes in vivo.

The above example demonstrates, among other things, that the methods of the present invention are suitable for use in a recipient host with cardiac tissue damage.

Therefore, the present invention is well adapted to attain the ends and advantages mentioned as well as those that are inherent therein. While numerous changes may be made by those skilled in the art, such changes are encompassed within the spirit of this invention as defined by the appended claims.

1. A method of stimulating cardiac tissue regeneration in a recipient host having cardiac tissue comprising administering peripheral blood derived stem cells to the host in an amount sufficient to produce stem cell-derived cardiomyocytes in the host's cardiac tissue.

2. The method of claim 1 further comprising administering an amount of peripheral blood derived stem cells sufficient to produce stem cell derived endothelial cells in the host's cardiac tissue.

3. The method of claim 1 further comprising administering an amount of peripheral blood derived stem cells sufficient to produce stem cell derived smooth muscle cells in the host's cardiac tissue.

4. The method of claim 1 wherein the peripheral blood derived stem cells are lineage committed.

5. The method of claim 1 wherein the peripheral blood derived stem cells are enriched based on a marker.

6. The method of claim 5 wherein the marker is CD34.

7. The method of claim 5 wherein the marker is CD133.

8. The method of claim 1 wherein the peripheral blood derived stem cells comprise G-CSF-mobilized human peripheral blood derived stem cells.

9. The method of claim 1 wherein the peripheral blood derived stem cells are from a human.

10. The method of claim 1 wherein the peripheral blood derived stem cells are autologous.

11. The method of claim 1 wherein administering comprises injecting the peripheral blood derived stem cells into the host's general circulation.

12. The method of claim 1 wherein administering comprises injecting the peripheral blood derived stem cells into the recipient host's heart.

13. The method of claim 1 wherein administering comprises injecting the peripheral blood derived stem cells into ischemic tissue, near ischemic tissue, or both.

14. The method of claim 1 wherein the host has suffered acute or chronic ischemia.

15. The method of claim 14 wherein the host has regeneration in the peri-infarct region.

16. The method of claim 1 wherein the host has previously received chemotherapy.

17. The method of claim 1 wherein the host has damaged cardiac tissue.

18. A method of regenerating cardiac tissue in a recipient host having cardiac tissue comprising:
obtaining peripheral blood from a donor;
enriching a sample derived from the peripheral blood for peripheral blood derived stem cells based on a marker;
transplanting into the recipient host the enriched sample, wherein the peripheral blood derived stem cells form cardiomyocytes in the host's cardiac tissue.

19. The method of claim 18 wherein the peripheral blood derived stem cells form new blood vessels in the cardiac tissue of the recipient host, thereby increasing the blood flow to the cardiac tissue.

20. The method of claim 18 wherein the peripheral blood derived stem cells are autologous.

21. A method of obtaining a differentiated cardiac cell comprising:
obtaining a stem cell from peripheral blood of a donor; and
providing the stem cell to a damaged cardiac tissue in a recipient host, wherein the stem cell differentiates into new cardiac cell.

22. The method of claim 21, wherein the peripheral blood derived stem cell is autologous.
